(19) United States
(12) Patent Application Publication
(10) Pub. No.: US 2012/0225054 A1
(43) Pub. Date: Sep. 6, 2012

(54) USE OF LEGUME EXTRACTS FOR INDUCING AND ENHANCING AUTOPHAGY AND APOPTOSIS AND FOR PREVENTING AND/OR TREATING CANCERS

(75) Inventors: FANG-NAN CHEN, CHIAYI COUNTY (TW); SHEN-JEU WON, CHIAYI COUNTY (TW); CHUN-LI SU, CHIAYI COUNTY (TW)

(73) Assignee: HERBAL BON BIOTECH INC., CHIAYI COUNTY (TW)

(21) Appl. No.: 13/038,656

(22) Filed: Mar. 2, 2011

(51) Int. Cl. A61K 36/48; A61P 25/28; A61P 1/16; A61P 35/00; A61P 3/10

(52) U.S. Cl. 424/115

(57) ABSTRACT

This invention relates to a use of a fermented Glycine max (L.) extract prepared by fermenting a Glycine max (L.) extract in inducing autophagy in a subject. In particular, the fermented Glycine max (L.) extract can be used in preventing and/or treating a disease in which in a subject, such as cancer, diabetes neurodegeneration, steatohepatitis and aging. The invention also relates to a use of the fermented Glycine max (L.) extract in inducing apoptosis in combination with autophagy in a subject.

### FIELD OF THE INVENTION

The present invention relates to use of a fermented legume extract (in particular, Glycine max (L.) extract) in inducing autophagy in a subject for treating or preventing a disease or disorder, such as diabetes, aging, neurodegeneration, steatohepatitis or cancer. The invention also relates to a use of the fermented legume extract (in particular, Glycine max (L.) extract) for selectively killing tumor cells by inducing cell apoptosis in combination with autophagy.

### BACKGROUND OF THE INVENTION

Apoptosis (type I) and autophagy (type II) are two different types of programmed cell death (Shimizu et al., 2004, Role of Bcl-2 family proteins in a non-apoptotic programmed cell death dependent on autophagy genes. Nat. Cell Biol. 6, 1221-1228). Apoptosis and autophagy are genetically regulated, evolutionarily-conserved processes that regulate cell fate; however, apoptosis invariably contributes to cancer cell death, whereas autophagy plays the Janus role of cancer cell survival and death. Apoptosis is an organized and energy-dependent process, which allows the organism to maintain tissue homeostasis. Insufficient of apoptosis contributes to the pathogenesis of cancer.

Autophagy is also a normal physiological process which promotes cell adaptation and survival, but under some conditions it leads to cell death (Lockshin and Zakeri, 2004, Int. J. Biochem. Cell Biol. 36, 2405-2419; Maiuri et al., 2007, Self-eating and self-killing: crosstalk between autophagy and apoptosis. Nat. Rev. Mol. Cell. Biol. 8, 741-752). Autophagy is an important mechanism for targeting cellular components including proteins, protein aggregates and organelles for degradation in lysosomes, and has been implicated in many diseases. The process of autophagy involves degradation and recycling of cell organelles and proteins in autolysosomes (a fusion compartment of autophagosomes and lysosomes). This catabolic, cellular self-digestion process is induced in response to starvation or stress, causing the formation of double membrane vesicles called autophagosomes that engulf proteins and organelles. Autophagosomes then fuse with lysosomes where the autophagosome and their cargo are degraded. This lysosome-mediated cellular self-digestion serves to recycle intracellular nutrients to sustain cell metabolism during starvation and to eliminate damaged proteins and organelles that accumulate during stress. Although elimination of individual proteins occurs by the ubiquitin-mediated proteasome degradation pathway, the autophagy pathway can eliminate protein aggregates and organelles. Thus, autophagy complements and overlaps with proteasome function to prevent the accumulation of damaged cellular components during starvation and stress. Through these functions, autophagy is an essential cellular stress response that maintains protein and organelle quality control, protects the genome from damage, and sustains cell and mammalian viability. Autophagy dysfunction is a major contributor to diseases including, but not limited to, age-associated diseases, neurodegeneration, steatohepatitisliver disease, diabetes and cancer. Many human neurodegenerative diseases are associated with aberrant mutant and/or polyubiquitinated protein accumulation and excessive neuronal cell death. Neurons of mice with targeted autophagy defects accumulate polyubiquitinated- and p62 containing protein aggregates that result in neurodegeneration. The human liver disease steatohepatitis and a major subset of hepatocellular carcinomas (HCCs) are associated with the formation of p62-containing protein aggregates, p62 is a common component of cytoplasmic inclusions in protein aggregation diseases. Livers of mice with autophagy defects have p62-containing protein aggregates, excessive cell death, and HCC. Recent findings have also shown that autophagy to be crucial for proper insulin secretion and β-cell viability.

Dietary intake of soybean and soybean-based products has been reported to reduce risks of several cancers. U.S. Patent Publication No. 2002/0182274 relates to therapeutic uses of fermented soy extracts in promoting general health, improving health of subjects, preventing and/or treating cancer, preventing infections, reducing incidence of infections, treating infections, treating asthma, treating inflammation, modulating immune system and treating immune disorders. U.S. Pat. No. 6,685,973 discloses a use of fermented Glycine max (L.) extract in inhibiting 15-lipooxygenase and preventing/treating a disease in which 15-lipooxygenase inhibition is implicated such as cardiovascular diseases, cancer, immune disorders and inflammation. In addition, a previous published report has revealed that SC-1, the filtered (0.22 μm) aqueous phase of soybean fermentation products by bacteria Bacillus subtilis and Bacillus brevis, significantly inhibited the growth and clonogenicity of HBV-related HCC Hep 3B cells and mouse hepatoma ML-1 cells (Su et al., 2007, Supernatant of bacterial fermented soybean induces apoptosis of human hepatocellular carcinoma Hep 3B cells via activation of caspase 8 and mitochondria. Food Chem. Toxicol. 45, 303-314). The report also suggests that article Cytotoxicity of SC-1 on cultured Hep 3B cells was due to the induction of caspase-8 and mitochondria-related apoptosis. However, none of prior art teaches and suggests the relationship between autophagy and the effect of soybean extract in induction and even enhancement of autophagy.

### SUMMARY OF THE INVENTION

The invention provides a method for inducing autophagy in a subject, which comprises administering to a subject an effective amount of a fermented Glycine max (L.) extract or a composition thereof.

The invention also provides a method for inducing autophagy and apoptosis in a subject, which comprises administering to a subject an effective amount of a fermented Glycine max (L.) extract or a composition thereof.

### BRIEF DESCRIPTION OF THE DRAWING

FIG. 1 shows growth of ML-1 cells in BALB/c mice. (A) Representative photograph of the mice bearing ML-1 cells after oral administration of vehicle (water, control group) or the fermented Glycine max (L.) extract (1.3 ml/mouse/day). SCB refers to unfiltrated fermented Glycine max (L.) extracts containing live bacteria. (B) Tumor volumes in the mice bearing ML-1 cells. Data are presented as means±SEM (n=4 each group). *: Significant different from the corresponding values of the vehicle control group.

FIG. 2 show the fermented Glycine max (L.) extract induced apoptosis of ML-1 cells implanted to BALB/c mice. SCB refers to unfiltrated fermented Glycine max (L.) extracts containing live bacteria. (A) Representative section of tumors viewed at 100×. (B) Representative section of tumors viewed at 400×. Sections of tumors were stained with Hoechst 33258 (blue) and fluorescein-dUTP (TUNEL assay; green) to visualize cell nuclei and apoptotic nuclei, respectively. Results are representative of three independent experiments.

FIG. 3 shows that fermented Glycine max (L.) extract induced autophagy of ML-1 cells implanted to BALB/c mice. SCB refers to unfiltrated fermented Glycine max (L.) extracts containing live bacteria. (A) Representative section of tumors viewed at 100×. (B) Representative section of tumors viewed at 400×. Sections of tumors were stained with Hoechst 33258 (blue) and anti-cleaved LC3 antibody (red) to visualize cell nuclei and autophagic punctate pattern of LC3-II respectively. Results are representative of three independent experiments.

FIG. 4 shows induction of both apoptosis and autophagy in tumors. SCB refers to unfiltrated fermented Glycine max (L.) extracts containing live bacteria. (A) Tumors obtained from the mice received oral administration of water (control group). (B) Tumors obtained from the mice received oral administration of the fermented Glycine max (L.) extract (1.3 ml/mouse/day). Sections of tumors were stained with Hoechst 33258 (blue), fluorescein-dUTP (TUNEL assay; green), and anti-cleaved LC3 antibody (red) to visualize cell nuclei, apoptotic nuclei, and autophagic punctate pattern of LC3-II, respectively. Results are representative of three independent experiments.

FIG. 5 shows effect of autophagy inhibitor on the fermented Glycine max (L.) extract-induced apoptosis. (A) Inhibitory effect of 3-MA on ML-1 cells. (B) Inhibitory effect of 3-MA on Hep 3B cells. After treatment, the cells were stained with propidium iodine for flow cytometry. The percentage in the figure indicates the proportion of apoptotic cells arrested at sub-G1 phase. The control cells (C) were cultured with DMEM. Results are representative of three independent experiments.

### DETAILED DESCRIPTION OF THE INVENTION

The invention unexpectedly found that the fermented legume extract (in particular, Glycine max (L.) extract) can effectively induce and even enhance autophagy in a subject. In particular, the said extract can induce autophagy and apotosis. Accordingly, the said extract has surprisingly effect in preventing and/or treating cancer.

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition.

As utilized in accordance with the present disclosure, the following terms, unless otherwise indicated, shall be understood to have the following meanings:

The term “apoptosis” as used herein refers to the process of cell death which results from an orderly pattern of morphological and biochemical changes.

The term “autophagy” as used herein refers to a catabolic process involving the degradation of a cell's own components through the lysosomal machinery and is a tightly-regulated process that plays a normal part in cell growth, development, and homeostasis, helping to maintain a balance between the synthesis, degradation, and subsequent recycling of cellular products.

The term “Glycine max (L.)” as used herein refers to a diploidized tetraploid (2n=40) plant, in the family Fabaceae, the genus Glycine Willd. and the subgenus Soja (Moench). The Glycine max (L.) preferred used in the preparation of the fermented Glycine max (L.) extract is selected from the group consisting of soybean and black soybean. The more preferably Glycine max (L.) is soybean.

The terms “a” and “an” refer to one or to more than one (i.e., to at least one) of the grammatical object of the article.

The term “treat,” “treatment” or “treating” means reducing the frequency, extent, severity and/or duration with which symptoms of cancer are experienced by a patient.

The term “prevent,” “prevention” or “preventing” means inhibition or the averting of symptoms associated with cancer.

The term “effective amount” means an amount of Glycine max (L.) extract effective to induce or enhance autophagy or antophagy-induced apoptosis.

In one aspect, the invention provides a method for inducing autophagy in a subject, which comprises administering to a subject an effective amount of a fermented Glycine max (L.) extract or a composition thereof. On the other hand, the invention provides a use of a fermented Glycine max (L.) extract in the manufacture of a medicament for inducing autophagy in a subject.

In another aspect, the invention provides a method for inducing autophagy and apoptosis in a subject, which comprises administering to a subject an effective amount of a fermented Glycine max (L.) extract or a composition thereof. On the other hand, the invention provides a use of a fermented Glycine max (L.) extract in the manufacture of a medicament for inducing autophagy-induced apoptosis in a subject.

According to the invention, the fermented Glycine max (L.) extract is prepared by fermenting an aqueous Glycine max (L.) extract with at least one bacterium. According to the invention, the preferred Glycine max (L.) used in the preparation of the fermented extract is soybean or black soybean. Preferably, the fermented Glycine max (L.) extract of the invention is the fermented soybean extract.

According to the invention, at least one bacterium is used in the fermentation of Glycine max (L.) extract. Preferably, the fermentation is carried out using a bacterium selected from the group consisting of Bacillus brevis, Bacillus subtilis, Bacillus stearothermophilus and Enterococcus faecium. More preferably, the bacterium used in the present invention is Bacillus brevis and/or Bacillus subtilis. In addition, lactic acid or yeast also can be used in the fermentation of the invention. When more than one bacterium is used in the fermentation, the fermentation can be conducted with the bacteria sequentially or simultaneously.

According to the invention, the fermentation is performed under conditions suitable for the growth of bacteria. Preferably, the temperature for fermentation ranges from 32° C. to 42° C. The fermentation time may be at least 5 days. Preferably, the time may be from 5 to 60 days; more preferably, the time is from 10 to 45 days. After fermentation, the fermented liquid is optionally sterilized, e.g. by heat or irradiation, preferably by heat, to obtain a sterilized liquid. Preferably, the sterilized liquid is filtered or centrifuged, preferably filtered, to remove most or all of the dead microbes to obtain the fermented Glycine max (L.) extract. More preferably, the filtration step is followed by removal of some of the water from the filtrate to concentrate the fermented liquid to obtain the fermented Glycine max (L.) extract.

According to the invention, the fermented Glycine max (L.) extract can induce autophagy. Anticancer therapies such as chemicals, irradiation, and hyperthermia induce autophagy and result in death (autophagic cell death) of breast, colon, prostate, and brain cancers. However, autophagy might remove the proteins or organelles that are damaged by cancer therapy, and become protective to the treatment (protective autophagy). The invention unexpectedly found that the fermented Glycine max (L.) extract can induce and even enhance autophagy. Accordingly, the fermented Glycine max (L.) extract can be used in the prevention and/or treatment of cancer, diabetes, neurodegeneration, steatohepatitis or aging.

Furthermore, the fermented Glycine max (L.) extract can induce autophagy and apoptosis. Preferably, the apoptosis is induced by the autophagy (autophagy induced-apoptosis). Induction of apoptosis and autophagy leads to the reduction in the growth of cancer cells and represents a balanced result between tumorigenic activities and immune responses. In addition, administration of the fermented Glycine max (L.) extract suppresses the growth of cancer cells via induction of apoptosis and autophagy without significant changing the body and liver weights, indicating the safety and efficacy of the fermented Glycine max (L.) extract in vivo and suggesting chemotherapeutic potential of the fermented Glycine max (L.) extract on cancers. Accordingly, the fermented Glycine max (L.) extract is effect in prevention and/or treatment of cancers. Preferably, the cancer is breast cancer, prostrate cancer, leukemia, colon cancer, uterine cancer, ovarian cancer, endometrial cancer, cervical cancer, colon cancer, testicular cancer, lymphoma, rhabdosarcoma, neuroblastoma, pancreatic cancer, lung cancer, brain tumor, skin cancer, gastric cancer, liver cancer, kidney cancer or nasopharyngeal carcinoma. More preferably, the cancer is liver cancer. Most preferably, the cancer is human hepatocellular carcinoma.

In this invention, the fermented Glycine max (L.) extract may be administered alone or in a composition comprising the fermented Glycine max (L.) extract and a pharmaceutically acceptable carrier, diluent and/or excipient. Preferably, the fermented Glycine max (L.) extract is fermented soybean extract or fermented black soybean extract. The fermented Glycine max (L.) extract may be administered at a dose of about 10⁻³ to 10 ml/kg body weight. Preferably, the dose of the fermented Glycine max (L.) extract is 0.01 to 3 ml/kg, more preferably 0.1 to 1 ml/kg, body weight. These doses are based on the fermented Glycine max (L.) extract in the concentrated form, but appropriate doses of the fermented Glycine max (L.) extract in the unconcentrated form or dry powder form can be calculated accordingly. The dose can be adjusted based on the health condition of the subject or the disease to be prevented or treated.

The fermented Glycine max (L.) extract was demonstrated to be highly safe for daily intake on a long-term basis in a 2 months chronic toxicity study of rodents. Mice receiving a dose of 1 ml/mouse and 1.3 ml/mouse for 60 days did not exhibit any significant difference or mean body weights or mean liver weights in an oral toxicity study. Signs of gross toxicity or mortality were never observed in tested animals.

The fermented Glycine max (L.) extracts of the present invention can optionally be in various formulations depending on the route of administration. Optional routes of administration are preferably, but not limited to topical and oral. Compositions for oral administration, can be in a form which includes powders or granules, suspensions, emulsions or solutions in water or non-aqueous media, oil or fat, sachets, capsules, tablets, gelcaps, food additives and sustained release formulations. A food additive containing fermented Glycine max (L.) extracts can optionally be used as an additive to edible oils in foods, such as but not limited to bread, baking products, biscuits, crackers, dairy products, cakes, chocolate and food fats.

Thickeners, diluents, flavorings, vitamins dispersing aids, emulsifiers or binders may be desirable. Preferred formulations for oral administration include gelatin capsules, preferably comprising ASU complex, vitamins, anti-oxidants and diluents. An additional preferred formulation for oral administration comprises ASU complex, vitamins, anti-oxidants and edible oil.

The following examples are provided to aid those skilled in the art in practicing the present invention. Even so, the examples should not be construed to unduly limit the present invention as modifications and variations in the embodiments discussed herein may be made by those having ordinary skill in the art without departing from the spirit or scope of the present inventive discovery.

### EXAMPLE

### Example 1

### Preparation of Soybean Fermentation Products

One kg of dried soybeans (Dongshi Shiang, Chiayi, Taiwan) were ground, boiled, and soaked in water for 10 days. After removing the big chuck of soybeans, the supernatant was fermented with Bacillus subtilis (10⁵ cells/ml) and Bacillus brevis (10⁵ cells/ml) for 1 month at 37° C. (Su et al., 2007).

### Example 2

### Efficacy Assays of the Fermented Soybean Extract

### Materials & Methods

### Cell Culture

Murine ML-1 cells obtained from Dr. Huan-Yao Lei (Department of Microbiology and Immunology, Medical College, National Cheng Kung University, Tainan, Taiwan) were cultured in complete Dulbecco's modified Eagle medium (DMEM; GIBCO BRL, Grand Island, N.Y.) containing 10% fetal bovine serum (GIBCO BRL), 2 mM glutamine (Sigma, St. Louis, Mo.), 100 U/ml penicillin (Sigma), and 100 μg/ml streptomycin (Sigma) at 37° C. in a 5% CO₂ humidified atmosphere.

### Animal Study

BALA/c mice at 6-7 weeks of age with body weight between 18-23 g were obtained from the Animal Center of the National Cheng Kung University (NCKU, Tainan, Taiwan). They were bred and housed at the Animal Center in a temperature-controlled and air-conditioned environment with a 10/14 h light/dark cycle. Food and water were provided ad libitum. ML-1 cells were implanted subcutaneously (s.c.; 2.5×10⁵ cells/mouse) to the flank of BALB/c mice at day 0. The mice were randomly divided into three groups at day 4. Treatment groups of mice received oral administration of the fermented soybean extracts (1.0 or 1.3 ml/mouse/day) for 56 consecutive days, and another group received vehicle (water) on the same schedule. Mice were monitored every other day for gross anatomical changes. Tumor growth was measured with a caliper every other day. Tumor volume was calculated by using the formula L×W²/2, where L (length) and W (width) are in millimeters and L is greater than W. All animal experiments were approved by the Animal Research Committee of NCKU and were performed under the guidelines of the National Research Council, Taiwan (IACUC940047).

### Immunohistochemistry

Tumors obtained from the fermented Glycine max (L.) extracts- or vehicle-treated mice were frozen on liquid nitrogen and stored at −80° C. until use. Apoptosis was detected using terminal deoxynucleotidyl transferase-mediated dUTP nick-end-labeling (TUNEL) method (ApoAlert DNA Fragmentation assay Kit, Clontech, Palo Alto, Calif.). Autophagy was examined using anti-cleaved LC3 antibody (ABGENT, San Diego, Calif.; 1:300). Tumor sections were subjected to apoptosis and/or autophagy analysis. Briefly, tumor sections (5 μm) cut by Cryotome0620 (Thermo Shandon, Waltham, Mass.) were incubated with 3.7% formaldehyde (Sigma) for 1 min at room temperature and then with cold ethanol (Merck, Darmstadt, Germany)/acetic acid (Wako, Osaka, Japan; 2:1, v/v) for 5 min at −20° C. Subsequently, the sections were quenched with 3% hydrogen peroxide (Wako) for 5 min, and subjected to TUNEL assay according to the manufacturer's protocol and the incorporated fluorescein-dUTP at the free 3′-hydroxyl ends of fragmented DNA was detected. After washing, the sections were incubated with blocking buffer (SuperBlock Blocking Buffer, Thermo Scientific, Rockford, Ill.) for 30 min at room temperature. Immunostaining was carried out by incubating tumor sections with rabbit polyclonal anti-cleaved LC3 antibody (ABGENT, San Diego, Calif.; 1:300) for overnight at 4° C. and then with goat anti-rabbit Alexa Fluor568-conjugated secondary antibody (Molecular Probes, Inc., Eugene, Oreg.; 1:450) in blocking solution for 2 h at room temperature. After washing, fluorescein-dUTP and/or anti-cleaved LC3 antibody stained sections were incubated with Hoechst 33258 (Sigma-Aldrich, St. Louis, Mo.; 0.05 μg/ml in PBS) for 10 min at room temperature. The signals were detected with a fluorescence microscope (OLYMPUS BX51).

### Flow Cytometric Analysis of Apoptotic Cells

Cells (2×10⁵) grown in 6-well plates were pretreated with or without 10 mM of autophagy inhibitor 3-methyladenine (3-MA; Sigma, St. Louis, Mo.) for 2 h prior to the addition of SC-1 (265 μg/ml). The cells were harvested, centrifuged at 800g for 10 min at 4° C., and resuspended in HBS containing 40 μg/ml of propidium iodide and 100 mg/ml RNase A for 30 min at 37° C. in the dark. Measurement of apoptotic cells was performed using a FACScan flow cytometer (Becton Dickison, Mountain View, Calif., USA).

### Statistical Analysis

The results were expressed as means±standard errors of the means (SEM). Differences in tumor volumes were analyzed by the Student's t test (Minitab software, version 10.2). A difference was considered if P<0.05. Weights of body, tumor, and liver were analyzed by One-way ANOVA. Differences among groups were analyzed by Duncan's multiple range test (SPSS software, version 14.0). A difference was considered if P<0.05.

### The Efficacy of Fermented Soybean Extract on the Growth of ML-1 Cells In Vivo

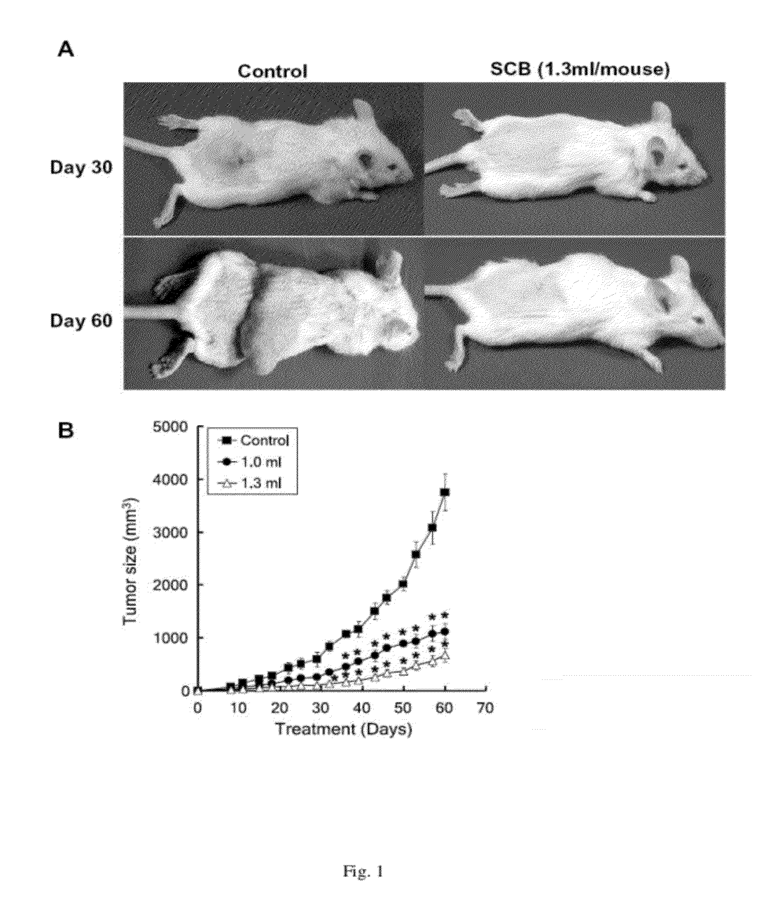

As described above, murine ML-1 cells were implanted s.c. to the flank of inbred BALA/c mice followed by oral administration of the fermented soybean extracts (1.0 or 1.3 ml/mouse/day) or vehicle (water) for 56 consecutive days. The growth of ML-1 cells was monitored every other day until day 60. As shown in FIG. 1A, at day 30, the growth of ML-1 cells was apparent in the control mice received vehicle. In contrast, the growth of ML-1 cells in the mice received the fermented soybean extract (1.3 ml/mouse/day) was not visible. At day 60, the size of the tumor in the control mice became much larger compared with that at day 30, whereas that in the group of mice received the fermented soybean extract (1.3 ml/mouse/day) was not dramatically changed. During the experiment, growth of tumor was measured with a caliper every other day. Differences in tumor volumes were analyzed. As shown in FIG. 1B, administration of the fermented soybean extracts (1.0 or 1.3 ml/mouse/day) significantly inhibited (P<0.05) the size of tumors throughout the experimental period. Of note, all mice survived until the end of the experiment. No apparent illness was found in the mice received the fermented soybean extracts. Body weights and liver weights were not significantly altered (P>0.05) by the fermented soybean extracts (Table 1).

TABLE 1
Effect of fermented soybean extract on the weights of
body, tumor, and liver

| | Body | Tumor | Liver |
|---|---|---|---|
| Treatment | Weight (g) | | |
| Control | 23.3 ± 0.7^{a} | 7.3 ± 1.3^{a} | 1.69 ± 0.16^{a} |
| SCB (1.0 ml/mouse) | 22.9 ± 0.7^{a} | 3.6 ± 0.7^{b} | 1.77 ± 0.04^{a} |
| SCB (1.3 ml/mouse) | 24.6 ± 0.6^{a} | 2.1 ± 0.2^{b} | 1.59 ± 0.05^{a} |
| P value | 0.146 | 0.005 | 0.474 |

Data are presented as means ± SEM (n = 4 each group).
SCB refers to unfiltrated fermented soybean extracts containing live bacteria.
Means within a column with different letters are significantly different, P < 0.05.

### The Efficacy of Fermented Soybean Extract in Inducing Apoptosis In Vivo

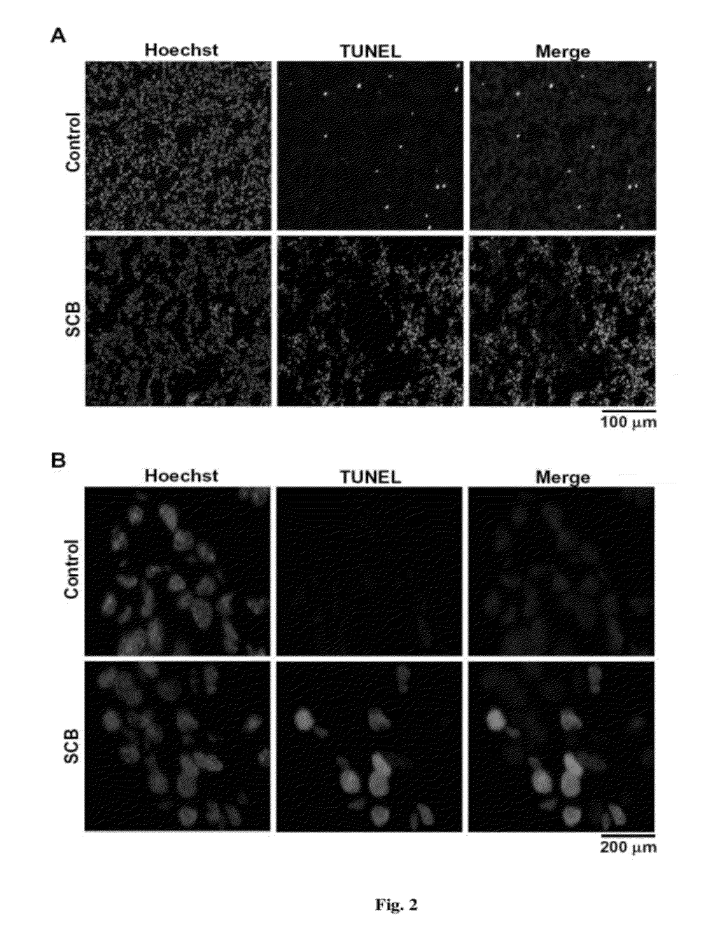

To confirm the induction of apoptosis in vivo, sections of tumors were subjected to TUNEL assay before fluorescence microscopy to examine the phenomenon of apoptosis, nuclear DNA double-strand breaks. As shown in FIG. 2A, viewed using fluorescence microscope at 100×, treatment of the fermented soybean extract increased positive TUNEL staining compared with the vehicle control. FIG. 2B, viewed at 400×, further reveals that the sections of tumors obtained from the mice received vehicle did not show apparent green apoptotic fluorescence at the nuclei, indicating that apoptotic events did not occur in the control. In contrast, the sections of tumors obtained from the mice received the fermented soybean extract (1.3 ml/mouse/day) exhibited intensive green fluorescence at the nuclei, representing nuclear DNA double-strand breaks, a hallmark of apoptosis.

### The Efficacy of Fermented Soybean Extract in Inducing Autophagy In Vivo

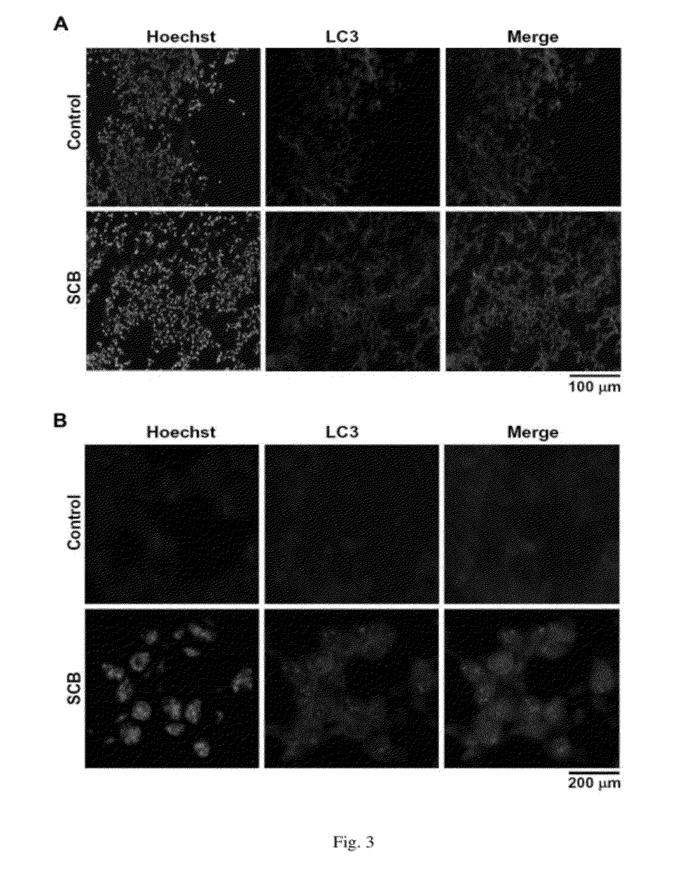

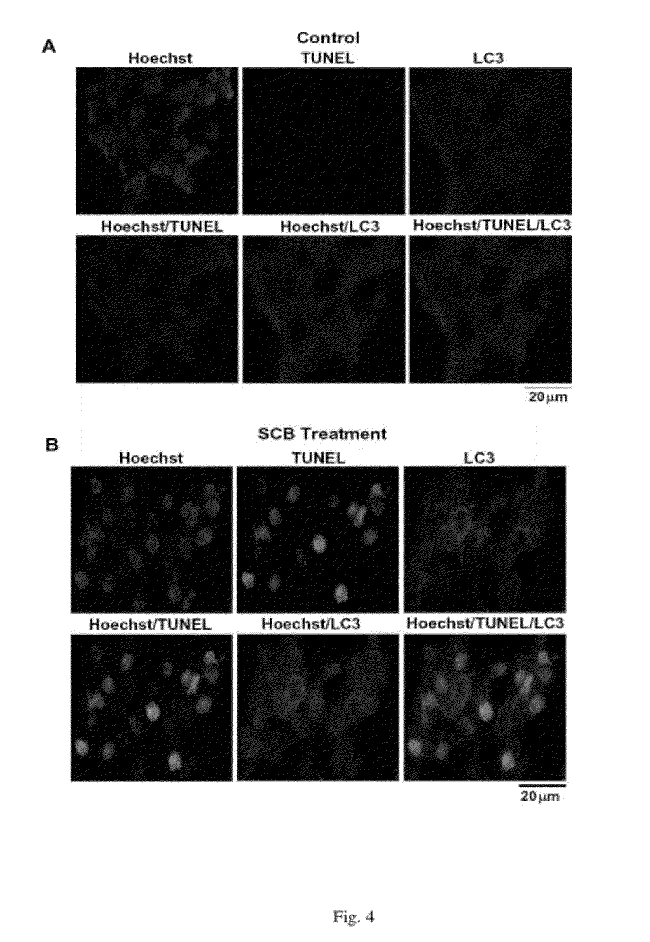

Phenomena of autophagy were determined in the tumors. As shown in FIG. 3A, morphometric analysis of cleaved LC3 distribution viewed at 100× exhibits that the fermented soybean extracts elevated cleaved LC3 staining compared with the control. Using fluorescence microscope viewed at 400× further discovered that no cytoplasmic cleaved LC3 was found around the nuclei in the control (FIG. 3B). In contrast, in the fermented soybean extracts-treated group, the expression of cleaved LC3 was increased and displayed a punctate staining, representing the location of autophagic marker LC3-II on autophogosomes. These results suggest that the fermented soybean extracts induced both apoptosis and autophagy in ML-1 cells in vivo. Furthermore, the merge images of Hoechst 33258, TUNEL, and cleaved LC3 staining indicates that the fermented soybean extracts induced autophagy alone without induction of apoptosis, characterized by blue nuclei surrounded with red LC3 puncta (FIGS. 4A and B). Interesting enough, the fermented soybean extracts did not induce apoptosis alone without induction of autophagy since almost every nucleus with green staining was surrounded with red cleaved LC3 puncta.

The Efficacy of Fermented Soybean Extract in Inducing Apoptosis in Combination with Autophagy

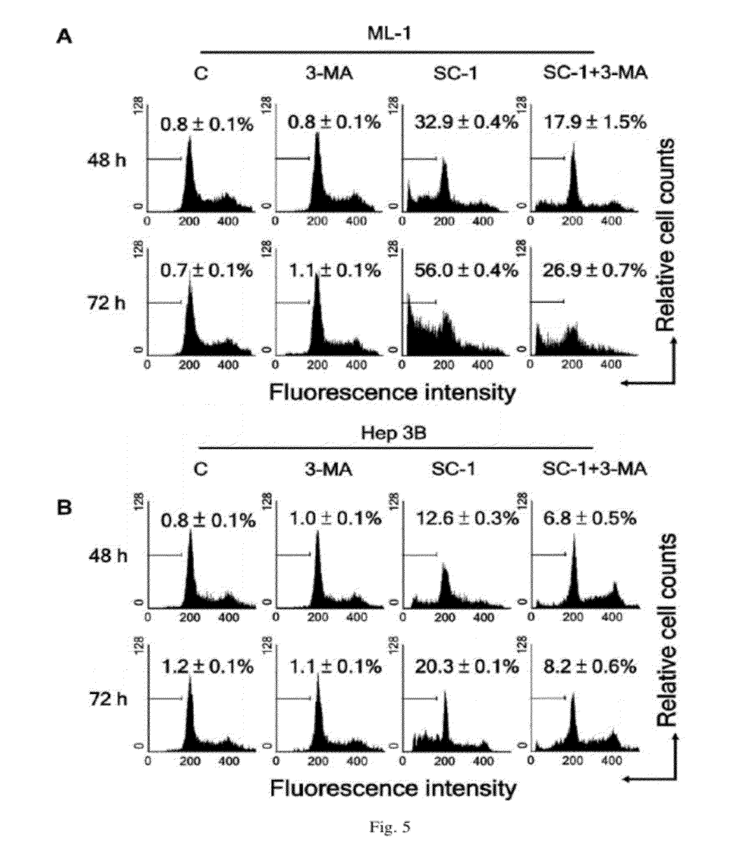

To confirm the importance of autophagy in the proceeding of apoptosis, autophagy inhibitor 3-MA was added to the cell culture and the relative portion of apoptotic cells was analyzed by flow cytometry. The results indicate that 3-MA suppressed the fermented soybean extracts-induced apoptosis in both murine hepatoma ML-1 and human HCC Hep 3B cells in a time-related manner. As shown in FIG. 5A, administration of 3-MA inhibited the fermented soybean extracts-induced apoptosis of ML-1 cells approximately 45% at 48 h and 52% at 72 h. In FIG. 5B, it inhibited the fermented soybean extracts-induced apoptosis of Hep 3B cells approximately 42% at 48 h and 60% at 72 h. Therefore, it would not be surprised that the induction of apoptosis was observed only in combination with autophagy in tumors obtained from the mice received the fermented soybean extracts for a relatively long period of time (FIGS. 4A and B).

What is claimed is:

1. A method for inducing autophagy in a subject, which comprises administering to a subject an effective amount of a fermented Glycine max (L.) extract or a composition thereof.

2. The method of claim 1, wherein the fermented Glycine max (L.) extract is prepared by fermenting an aqueous Glycine max (L.) extract with at least one bacterium.

3. The method of claim 2, wherein bacterium is selected from the group consisting of Bacillus brevis, Bacillus subtilis, Bacillus stearothermophilus and Enterococcus faecium.

4. The method of claim 2, wherein the bacterium is Bacillus brevis and/or Bacillus subtilis

5. The method of claim 1, wherein the fermented Glycine max (L.) extract is the fermented soybean or black bean extract.

6. The method of claim 1, wherein fermented Glycine max (L.) extract or a composition thereof can be used in the prevention and/or treatment of cancer, diabetes, neurodegeneration, steatohepatitis or aging.

7. The method of claim 6, wherein the cancer is breast cancer, prostrate cancer, leukemia, colon cancer, uterine cancer, ovarian cancer, endometrial cancer, cervical cancer, colon cancer, testicular cancer, lymphoma, rhabdosarcoma, neuroblastoma, pancreatic cancer, lung cancer, brain tumor, skin cancer, gastric cancer, liver cancer, kidney cancer or nasopharyngeal carcinoma.

8. The method of claim 6, wherein the cancer is liver cancer.

9. The method of claim 6, wherein the cancer is human hepatocellular carcinoma

10. A method for inducing autophagy and apoptosis in a subject, which comprises administering to a subject an effective amount of a fermented Glycine max (L.) extract or a composition thereof.

11. The method of claim 10, wherein the apoptosis is induced by autophagy.

12. The method of claim 10, wherein the fermented Glycine max (L.) extract is prepared by fermenting an aqueous Glycine max (L.) extract with at least one bacterium.

13. The method of claim 12, wherein bacterium is selected from the group consisting of Bacillus brevis, Bacillus subtilis, Bacillus stearothermophilus and Enterococcus faecium.

14. The method of claim 12, wherein the bacterium is Bacillus brevis and/or Bacillus subtilis

15. The method of claim 10, wherein the fermented Glycine max (L.) extract is the fermented soybean or black bean extract.

16. The method of claim 10 wherein fermented Glycine max (L.) extract or a composition thereof can be used in the prevention and/or treatment of cancer.

17. The method of claim 16, wherein the cancer is breast cancer, prostrate cancer, leukemia, colon cancer, uterine cancer, ovarian cancer, endometrial cancer, cervical cancer, colon cancer, testicular cancer, lymphoma, rhabdosarcoma, neuroblastoma, pancreatic cancer, lung cancer, brain tumor, skin cancer, gastric cancer, liver cancer, kidney cancer or nasopharyngeal carcinoma.

18. The method of claim 16, wherein the cancer is liver cancer.

19. The method of claim 16, wherein the cancer is human hepatocellular carcinoma
